(19) United States
(12) Patent Application Publication
(10) Pub. No.: US 2025/0366999 A1
(43) Pub. Date: Dec. 4, 2025

(54) BONE REPAIR SYSTEMS, METHODS, AND DEVICES

(71) Applicant: Surgical Device Innovations, LLC, Dallas, TX (US)

(72) Inventors: Jorge Casas-Ganem, Dallas, TX (US); Joshua Haney, Frisco, TX (US)

(73) Assignee: Surgical Device Innovations, LLC, Dallas, TX (US)

(21) Appl. No.: 19/097,625

(22) Filed: Apr. 1, 2025

Related U.S. Application Data
(60) Provisional application No. 63/655,261, filed on Jun. 3, 2024.

(51) Int. Cl. A61F 2/40; A61F 2/30

(52) U.S. Cl. CPC A61F 2/4014; A61F 2/30749; A61F 2/30771; A61F 2002/30326; A61F 2002/30331; A61F 2002/30528; A61F 2002/30616; A61F 2002/30784; A61F 2002/30851; A61F 2002/3092; A61F 2002/4011; A61F 2002/4022; A61F 2002/4037; A61F 2002/4044; A61F 2002/4074

(57) ABSTRACT

Systems, methods, and devices include a bone repair system with a modular configuration of components. A bone repair device includes a head which tapers from a circular face to a first connector stem. The bone repair device also includes a plurality of interchangeable middle portions. A middle portion includes a first receiving area at a first end and a second connector stem at a second end. The first receiving area is operable to engage the first connector stem of the head portion. The bone repair device further includes a stem portion including a second receiving area at a first end and a tapered implantation stem at a second end. The second receiving area is operable to engage the second connector stem of the middle portion. Furthermore, the stem portion, the middle portion, and the head portion are selectively coupled to form a modular arrangement.

### CROSS-REFERENCE TO RELATED APPLICATION

This application claims priority to U.S. Provisional Application Ser. No. 63/655,261, filed Jun. 3, 2024 and titled “BONE REPAIR SYSTEMS, METHODS, AND DEVICES,” the entirety of which is incorporated by reference herein.

### BACKGROUND

The current standard of care for proximal humerus defects involves a composite construct combining a cadaver bone combined with a shoulder replacement. Allografts are expensive and have a high infection risk. Surgical time with this technique is extremely long, further increasing infection risk. The benefit of a cadaveric bone graft is the ability to repair the patient's native rotator cuff to the cuff on the grafted bone. Proximal humerus replacement systems often result in shoulder instability and dismal functional outcomes.

It is with these observations in mind, among others, that various aspects of the presently disclosed technology were conceived and developed.

### BRIEF DESCRIPTION OF THE DRAWINGS

The foregoing summary, as well as the following detailed description, will be better understood when read in conjunction with the appended drawings. For the purpose of illustration, there is shown in the drawings certain examples of the disclosed subject matter. It should be understood, however, that the disclosed subject matter is not limited to the precise implementations and features shown. The accompanying drawings, which are incorporated in and constitute a part of this specification, illustrate implementations of systems, methods, and devices consistent with the disclosed subject matter and, together with the description, serves to explain advantages and principles consistent with the disclosed subject matter, in which:

FIG. 1A illustrates an example bone repair system including modular components of a bone repair implant;

FIG. 1B illustrates an example bone repair system including a head portion and a reverse total shoulder liner;

FIG. 2A illustrates an example bone repair system including a plurality of interchangeable middle portions;

FIG. 2B illustrates an example bone repair system including a plurality of interchangeable stem portions; and

FIG. 3 illustrates an example method of bone repair which can be performed by any of the systems depicted in FIGS. 1A-2B.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present technological concepts.

Systems, methods, and devices include a bone repair system with a modular implant device having lower profile and incorporating trabecular metal into the proximal body of the implant. The implant disclosed herein can have multiple points for soft tissue attachment. For instance, ingrowth of soft tissue into the trabecular metal portions can be superior to ingrowth into a roughened surface. Although some system can use roughened surfaces, ingrowth of the soft tissues onto these surfaces can be unpredictable and can result in a high failure rate, with avulsion of the soft tissues from the metal, resulting in shoulder instability or dislocation. As such, systems disclosed herein can use a particular arrangement of trabecular metal portions rather than or in addition to roughened surfaces.

In some scenarios, the systems disclosed herein can have a modular, low profile, with a trabecular metal proximal body that provides primary repair of the patient's native rotator cuff and shoulder joint capsule. This can maintain stability of the shoulder joint and can prevent pain, instability, and dislocation or subluxation. The bone repair implant can have a neck-shaft angle of 135 degrees, and/or between 125 degrees and 145 degrees, and a lateralized glenosphere, which can increase range of motion for the patient.

The system(s) disclosed herein can address the dilemma of post operative shoulder instability and dislocation that occurs in other systems. The disclosed system(s) can reduce and/or eliminate the wait time and expense associated with reconstruction with a cadaver graft. Advantages provided by the system(s) disclosed herein can include a lower profile, better soft tissue attachments, and/or restoration of shoulder stability and better functional outcomes. Furthermore, advantages over cadaver bone graft-prosthesis composite can include no wait time for cadaver grafts, no need to size match bone graft with patient, lower infection risk, a much shorter surgical time, and/or reproducibility of the reconstruction.

Additional advantages of the systems, methods, and devices discussed herein will become apparent from the detailed description below.

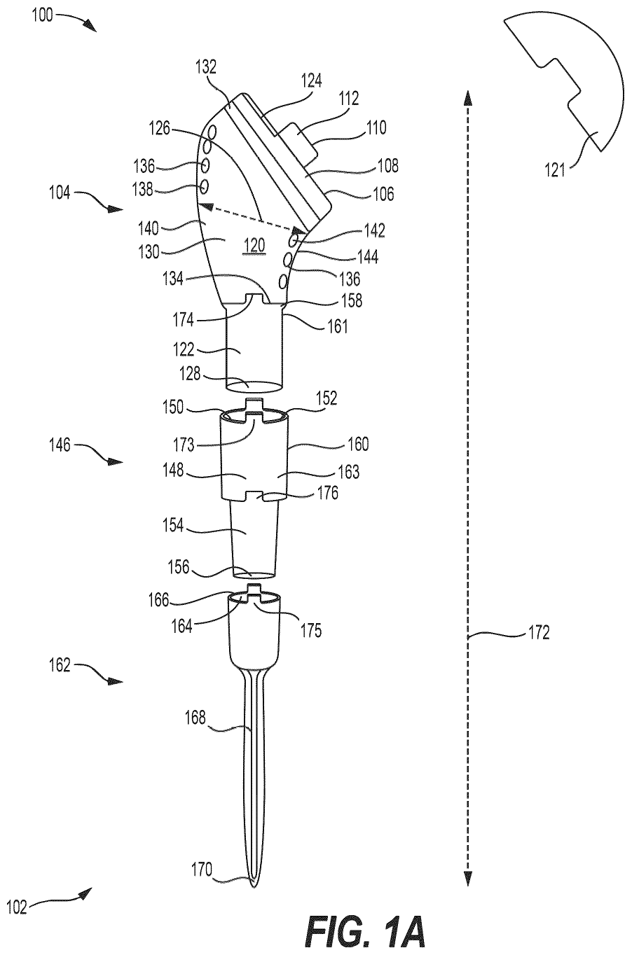

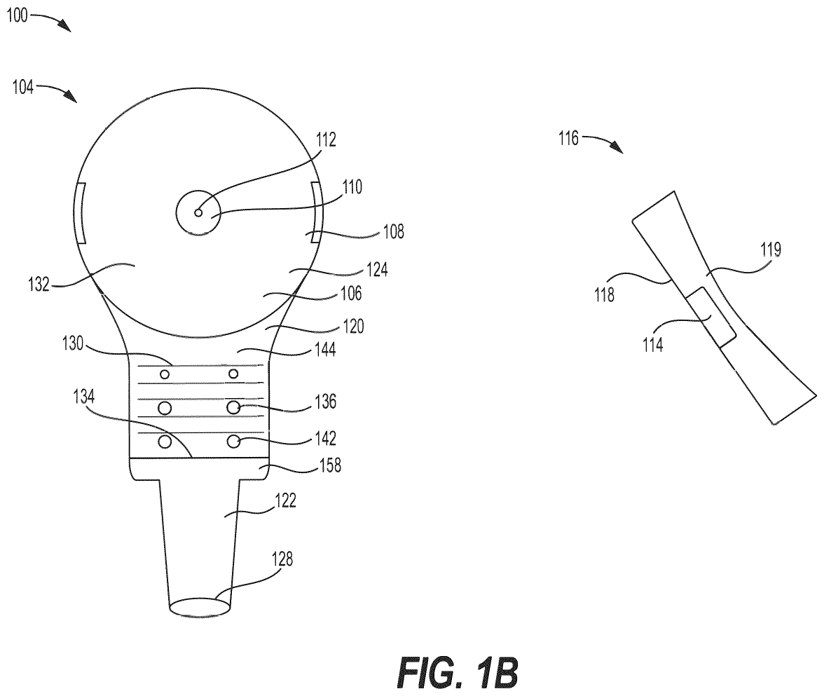

FIGS. 1A and 1B illustrate an example bone repair system 100 including a bone repair device 102 formed of multiple, modular components. For instance, the bone repair device 102 can include a head portion 104 with a circular face 106. The circular face 106 can have a flat surface 108 with a coupler 110 for engaging and coupling to a reverse total shoulder liner 116. The coupler 110 can be a protrusion 112 (e.g., a circular protrusion, a square protrusion, a triangle protrusion, etc.) which extends from a center of the circular face 106. The protrusion 112 can correspond to an indent 114 formed into the reverse total shoulder liner 116 such that the indent 114 is operable to receive the protrusion 112 when the reverse total shoulder liner 116 is mated to the circular face 106. For instance, the indent 114 can be formed into a center of a back surface 118 of the reverse total shoulder liner 116. Additionally or alternatively, the positions of the protrusion 112 and the indent 114 can be reversed, such that the protrusion 112 extends from the back surface 118 to correspond to the indent 114 on the circular face 106. The reverse total shoulder liner 116 can have a disk shape with a flat back surface 118 and/or a curved (e.g., concave) front surface 119, which can form a humeral cap operable for engaging a glenoid sphere. By way of example, the reverse total shoulder liner 116 can be formed of polyethylene, metal, composite materials, or combinations thereof. Using this configuration, the bone repair device 102 can selectively be used to mate with a cuff tear arthropathy head 121 and/or a glenoid sphere.

Furthermore, the head portion 104 can include a head portion body 120 which tapers from the circular face 106 to a first connector stem 122. For instance, the circular face 106 can define a first end 124 of the head portion body 120 with a greater width dimension 126 than a second end 128 defined by the first connector stem 122. The head portion body 120 can also include a first trabecular metal section 130 which extends from a circular disk section 132 at the first end 124 to a lower edge 134 of the first connector stem 122. The first trabecular metal section 130 can be metal with a porous and/or metallic fibrous structure, which can be manufactured using a 3D printing procedure, a machining procedure, a chemical wash procedure, and/or combinations thereof. Furthermore, the head portion body 120 can include one or more suture holes 136, such as a first set of suture holes 138 formed onto on outer side 140 of the head portion body 120 and/or a second set of suture holes 142 formed onto an inner side 144 of the head portion body. The first set of suture holes 138 and/or the second set of suture holes 142 can both include two, three, four, five, six, seven, eight, nine, or ten suture holes. The first set of suture holes 138 and the second set of suture holes 142 can include a same number of suture holes 136 or a different number of suture holes 136. In some scenarios, the one or more suture holes 136 can, additionally or alternatively, function as screw holes (e.g., by including a threading).

In some examples, the bone repair device 102 includes a middle portion 146. The middle portion 146 can have an elongated body 148 with a first receiving area 150 defining a first end 152 and a second connector stem 154 defining a second end 156. The first receiving area 150 can include an opening and/or indent formed into the first end 152 operable to receive and/or mate with the first connector stem 122 of the head portion 104. For instance, the first connector stem 122 can have an outer lip 158 having a similar or same width dimension as the first end 152 of the middle portion 146, such that the head portion 104 and the middle portion 146 form a continuous outer surface when mated together. In some scenarios, the middle portion 146 can include a second trabecular metal section 160 (e.g., a porous and/or metallic fibrous section) which can form a continuous trabecular metal section with the first trabecular metal section 130 when the head portion 104 is mated with the middle portion 146. Furthermore, the middle portion 146 can have a tapered body such that a width dimension at the first end 152 is greater than a width dimension at the second trabecular metal section 160. Moreover, the head portion 104 and/or the middle portion 146 can include a first morse taper 161 and/or a second morse taper 163 formed into the second end 128 of the head portion 104 and the second end 156 of the middle portion 146, respectively.

In some examples, the bone repair device 102 can include a stem portion 162 with a second receiving area 164 defining a first end 166 from which a tapered implantation stem 168 extends to define a second end 170. The second receiving area 164 can include an opening and/or indent formed into the first end 166 operable to receive and/or mate with the second connector stem 154 of the middle portion 146. The tapered implantation stem 168 can be an extension that tapers to a sharp point operable for insertion into a marrow of a bone, such that the bone repair device 102 forms a prosthetic bone portion (e.g., a prosthetic humerus portion) when implanted. Moreover, the head portion 104, the middle portion 146, and/or the stem portion 162 can be selected from a plurality of interchangeable components to selectively determine a length dimension 172 to match an anatomical dimension of the patient receiving the bone repair device 102.

In some instances, the bone repair device 102 can include one or more anti-rotational flanges on the modular components (e.g., the intercalary bodies, the stem, and/or the proximal humeral body). For example, the middle portion 146 can include a first anti-rotational flange 173 extending from the first end 152 (e.g., along an outer edge). The first anti-rotational flange 173 can be a fin, pin, and/or other type of protrusion with a shape corresponding to a shape of a first receiving cut-out 174 formed into the lower edge 134 of the head portion 104. The first anti-rotational flange 173 can have a square, rectangular, and/or trapezoidal shape which mates with a corresponding square, rectangular, and/or trapezoidal shape of the first receiving cut-out 174. Additionally, the first anti-rotational flange 173 can have another shape, such as a round shape, a pin-shape, a latch shape, or so forth to correspond to the shape of the first receiving cut-out 174. It is to be understood that the arrangement of the first anti-rotational flange 173 and the first receiving cut-out 174 can be reversed, such that the first anti-rotational flange 173 is disposed on the lower edge 134 of the head portion 104 and the first receiving cut-out 174 is disposed on the first end 152 of the middle portion 146. Furthermore, the first anti-rotational flange 173 and the first receiving cut-out 174 can include a plurality of first anti-rotational flanges 173 corresponding to a plurality first receiving cut-outs 174. For instance, two anti-rotational flanges 173 may protrude from opposite sides of the middle portion 146. Moreover, the first anti-rotational flanges 173 can include three, four, or any number of protrusions extending from the middle portion 146. Furthermore, anti-rotational flanges 173 can be formed into the interior of the first receiving area 150 for mating with receiving cut-outs 174 formed on the first connector stem 122. In this way, upon mating the head portion 104 with the middle portion 146, rotational movement of the head portion 104 relative to the middle portion 146 can be prevented by the interlocking function of the first anti-rotational flange 173 with the first receiving cut-out 174.

Similarly, in some examples, a second anti-rotational flange 175 can extend from the outer edge of the first end 166 of the stem portion 162. The second anti-rotational flange 175 can also be a fin, pin, and/or other type of protrusion with a shape corresponding to a shape of a second receiving cut-out 176 formed into the lower edge of the head portion 146. The second anti-rotational flange 175 can have a square, rectangular, and/or trapezoidal shape which mates with a corresponding square, rectangular, and/or trapezoidal shape of the second receiving cut-out 176. Additionally, the second anti-rotational flange 175 can have another shape, such as a round shape, a pin-shape, latch shape or so forth to correspond to the second receiving cut-out 176. It is to be understood that the arrangement of the second anti-rotational flange 175 and the second receiving cut-out 176 can be reversed, in that the second anti-rotational flange 175 can be disposed on the lower edge of the head portion middle portion 146 and the second receiving cut-out 176 can be disposed on the first end 166 of the stem portion 162. Furthermore, the second anti-rotational flange 175 and the second receiving cut-out 176 can include a plurality of second anti-rotational flanges 175 corresponding to a plurality of second receiving cut-outs 176. For instance, two anti-rotational flanges 175 may protrude from opposite sides of the stem portion 162. Moreover, the second anti-rotational flange 175 can include three, four, or any number of protrusions extending from the stem portion 162. Furthermore, anti-rotational flanges can be formed into the interior of the second receiving area 164 for mating with receiving cut-outs formed on the second connector stem 154. In this way, upon mating the head portion middle portion 146 with the stem portion 162, rotational movement of the middle portion 146 relative to the stem portion 162 can be prevented by the interlocking function of the second anti-rotational flange 175 with the cut-out 176.

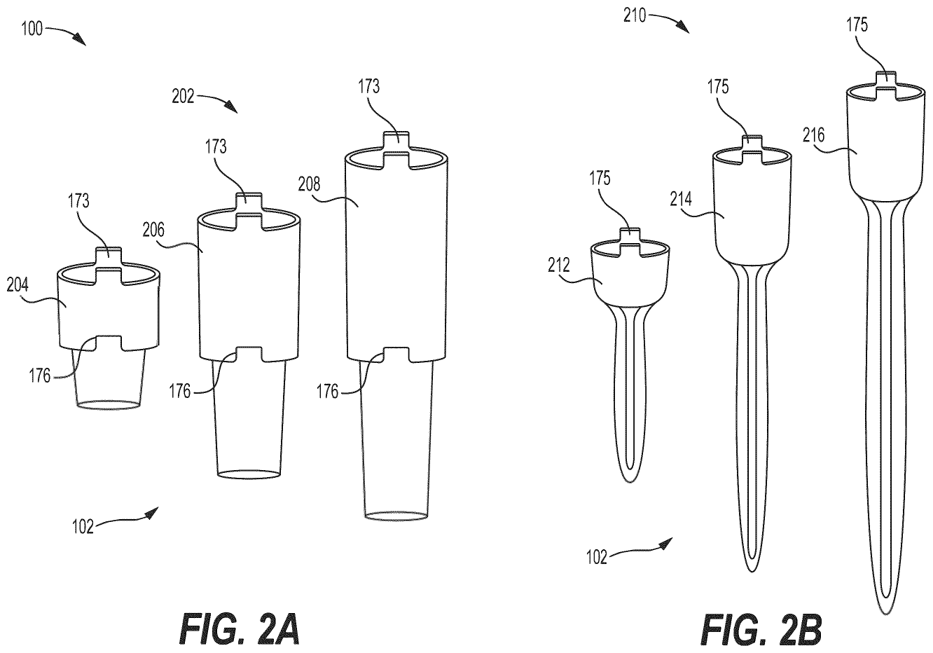

Turning to FIGS. 2A and 2B, the bone repair system 100 including the bone repair device 102 is depicted. As shown in FIGS. 2A and 2B, the bone repair system 100 can include a modular arrangement in which a plurality of interchangeable components can be used to selectively control the length dimension 172 of the bone repair device 102.

For instance, FIG. 2A depicts a plurality of interchangeable middle portions 202. The plurality of interchangeable middle portions 202 can include a first middle portion 204, a second middle portion 206, a third middle portion 208, and so forth. Each of the plurality of interchangeable middle portions 202 can have different length dimensions. For example, the first middle portion 204 can have a lesser length dimension than the second middle portion 206, the second middle portion 206 can had a lesser length dimension than the third middle portion 208, and so forth. Moreover, the plurality of interchangeable middle portions 202 can have a same width dimension and/or the same configurations of the first receiving areas 150 and the second connector stems 154, such that the plurality of interchangeable middle portions 202 can be interchangeable with each other for adjusting the length dimension 172 of the bone repair device 102.

Additionally, FIG. 2B depicts a plurality of interchangeable stem portions 210. The plurality of interchangeable stem portions 210 can include a first stem portion 212, a second stem portion 214, a third stem portion 216, and so forth. Each of the plurality of interchangeable stem portions 210 can have different length dimensions. For example, the first stem portion 212 can have a lesser length dimension than the second stem portion 214, the second stem portion 214 can have a lesser length dimension than the third stem portion 216, and so forth. Moreover, the plurality of interchangeable stem portions 210 can have a same width dimension and/or the same configurations of the second receiving area 164, such that the plurality of interchangeable stem portions 210 care interchangeable with each other for adjusting the length dimension 172 of the bone repair device 102.

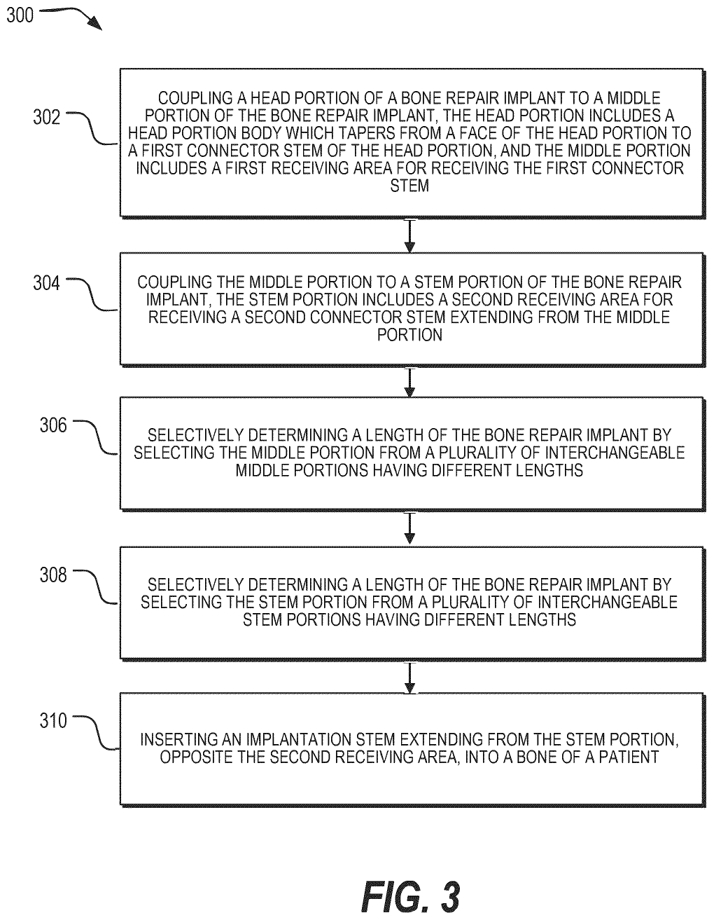

Turning to FIG. 3, an example method 300 of bone repair using a modular bone repair system is depicted. The method 300 can be performed by the system(s) and device(s) discussed above regarding FIGS. 1A-2B.

In some examples, at operation 302, the method 300 can couple a head portion of a bone repair implant to a middle portion of the bone repair implant, the head portion including a head portion body which tapers from a face of the head portion to a first connector stem of the head portion, and the middle portion includes a first receiving area for receiving the first connector stem. At operation 304, the method 300 can couple the middle portion to a stem portion of the bone repair implant, the stem portion including a second receiving area for receiving a second connector stem extending from the middle portion. At operation 306, the method 300 can selectively determine a length of the bone repair implant by selecting the middle portion from a plurality of interchangeable middle portions having different lengths. At operation 308, the method 300 can selectively determine a length of the bone repair implant by selecting the stem portion from a plurality of interchangeable stem portions having different lengths. At operation 310, the method 300 can insert an implantation stem extending from the stem portion, opposite the second receiving area, into a bone of a patient.

It is to be understood that the specific order or hierarchy of steps in the method(s) depicted in FIG. 3 and throughout this disclosure are instances of example approaches and can be rearranged while remaining within the disclosed subject matter. For instance, any of the operations depicted in FIG. 3 and throughout this disclosure may be omitted, repeated, performed in parallel, performed in a different order, and/or combined with any other of the operations depicted in FIG. 3 and throughout this disclosure.

While the presently disclosed technology has been described with reference to various implementations, it will be understood that these implementations are illustrative and that the scope of the presently disclosed technology is not limited to them. Many variations, modifications, additions, and improvements are possible. More generally, implementations in accordance with the presently disclosed technology have been described in the context of particular implementations. Functionality may be separated or combined differently in various implementations of the disclosure or described with different terminology. These and other variations, modifications, additions, and improvements may fall within the scope of the disclosure.

What is claimed is:

1. A bone repair system including:
a head portion including a circular face and a head portion body which tapers from the circular face to a first connector stem;
a plurality of interchangeable middle portions, a middle portion of the plurality of interchangeable middle portions includes a first receiving area at a first end and a second connector stem at a second end, the first receiving area being operable to engage the first connector stem of the head portion; and
a stem portion including a second receiving area at a first end and a tapered implantation stem at a second end, the second receiving area being operable to engage the second connector stem of the middle portion,
wherein,
the stem portion, the middle portion, and the head portion are selectively coupled to form a modular arrangement.

2. The bone repair system of claim 1,
wherein,
the plurality of interchangeable middle portions has different length dimensions for selectively determining a length of the modular arrangement.

3. The bone repair system of claim 1,
wherein,
the stem portion is one of a plurality of interchangeable stem portions having different length dimensions for selectively determining a length of the modular arrangement.

4. The bone repair system of claim 1,
wherein,
the head portion body includes a trabecular metal section between the circular face and the first connector stem.

5. The bone repair system of claim 1,
wherein,
the head portion body includes a plurality of suture holes.

6. The bone repair system of claim 5,
wherein,
the plurality of suture holes includes a first set of suture holes on an outer side of the head portion body and a second set of suture holes on an inner side of the head portion body.

7. The bone repair system of claim 4,
wherein,
the middle portion includes a trabecular metal section between the first receiving area and the second connector stem.

8. The bone repair system of claim 1,
wherein,
the head portion body includes one or more screw holes formed into an inner side.

9. The bone repair system of claim 1, further comprising:
a reverse total shoulder liner with a coupler operable to mate with the circular face.

10. The bone repair system of claim 9,
wherein,
the reverse total shoulder liner has a flat mating face, opposite a concave surface, operable to mate with the circular face.

11. The bone repair system of claim 10,
wherein,
the reverse total shoulder liner is at least partly formed of polyethylene.

12. The bone repair system of claim 10,
wherein,
the coupler includes an indent which corresponds to a protrusion extending from a center of the circular face.

13. A bone repair system comprising:
a head portion including a face with a protrusion or an indent operable to mate with a reverse total shoulder liner, and the head portion includes a head portion body which tapers from the face to a first connector stem;
a middle portion including a first receiving area at a first end and a second connector stem at a second end, the first receiving area being operable to engage the first connector stem of the head portion; and
a stem portion of a plurality of interchangeable stem portions, the stem portion includes a second receiving area at a first end and a tapered implantation stem at a second end, the second receiving area being operable to engage the second connector stem of the middle portion,
wherein,
the stem portion, the middle portion, and the head portion are selectively coupled to form a modular arrangement.

14. The bone repair system of claim 13, further comprising:
one or more ant-rotational flanges formed into the head portion, the middle portion, or the stem portion to prevent rotational movement of the middle portion relative to the head portion or the stem portion.

15. The bone repair system of claim 13, further comprising:
a first morse taper formed onto the first connector stem and corresponding to the first receiving area; and
a second morse taper formed into the second connector stem and corresponding to the second receiving area.

16. The bone repair system of claim 13, further comprising:
a first trabecular metal section formed into the head portion; and
a second trabecular metal section formed into the middle portion such that the first trabecular metal section is adjacent to the second trabecular metal section to form a continuous trabecular metal section when the head portion is coupled to the middle portion.

17. The bone repair system of claim 16,
wherein,
the head portion includes a longitudinal axis which forms a neck-shaft angle with a face axis, the face axis being perpendicular to the face, and the neck-shaft angle being between 125 degrees and 145 degrees.

18. A method of bone repair, the method comprising:
coupling a head portion of a bone repair implant to a middle portion of the bone repair implant, the head portion includes a head portion body which tapers from a face of the head portion to a first connector stem of the head portion, and the middle portion includes a first receiving area for receiving the first connector stem;
coupling the middle portion to a stem portion of the bone repair implant, the stem portion includes a second receiving area for receiving a second connector stem extending from the middle portion; and
inserting an implantation stem extending from the stem portion, opposite the second receiving area, into a bone of a patient.

19. The method of claim 18, further comprising:
selectively determining a length of the bone repair implant by selecting the middle portion from a plurality of interchangeable middle portions having different lengths.

20. The method of claim 18, further comprising:
selectively determining a length of the bone repair implant by selecting the stem portion from a plurality of interchangeable stem portions having different lengths.
